(19) United States
(12) Patent Application Publication
(10) Pub. No.: US 2022/0251279 A1
(43) Pub. Date: Aug. 11, 2022

(54) Method for Preparation of Acetoacetate Ester Compounds

(71) Applicant: Arxada AG, Visp (CH)

(72) Inventors: Maximilian Kuehnle, Rheinfelden (CH); Masaki Nakajima, Basel (CH); Andreas Breuer, Saas-Almagell (CH); Bruno Rittiner, Brig-Glis (CH); Nikolaus Bieler, Brig-Glis (CH)

(21) Appl. No.: 17/624,597

(22) PCT Filed: Jul. 3, 2020
(86) PCT No.: PCT/EP2020/068843

§ 371 Date: Jan. 4, 2022

(30) Foreign Application Priority Data
Jul. 5, 2019 (EP) 19184758.1
Jul. 30, 2019 (EP) 19189118.3

(51) Int. Cl. C08G 12/00; C07C 219/06; C07D 295/088; C08J 5/18; C09D 161/00; C09J 161/00

(52) U.S. Cl. CPC C08G 12/00; C07C 219/06; C07D 295/088; C08J 2361/00; C09D 161/00; C09J 161/00; C08J 5/18

(57) ABSTRACT

The invention discloses a method for preparation of acetoacetate ester compounds with amino alcohol catalysts which are themselves diketenized in the course of the diketenization reaction; and subsequent polymerization of these acetoacetate ester compounds with e.g. acrylate compounds to provide resins, whereby the acetoacetylated amino alcohol catalyst partakes in the polymerization reaction.

The invention discloses a method for preparation of acetoacetate ester compounds with amino alcohol catalysts which are themselves diketenized in the course of the diketenization reaction; and subsequent polymerization of these acetoacetate ester compounds with e.g. acrylate compounds to provide resins, whereby the acetoacetylated amino alcohol catalyst partakes in the polymerization reaction.

### BACKGROUND OF THE INVENTION

Polyurethane plastics, produced from multifunctional isocyanates such as 4,4′-Methylenediphenyl Isocyanates (MDI) or Toluene Diisocyanates (TDI) are used in many different applications, such as of sealants, adhesives, foams, coatings or as potting compounds due to their excellent properties.

Polyurethane resin compositions can fulfil many requirements of various products, such as good elasticity, excellent chemical resistance, excellent cost effectiveness, good thermal stability, good insulation and good processing properties. Especially the fast curing of the raw materials, the Polyol and the Isocyanate, is of special interest for many of these applications.

However, one of the main disadvantage of Isocyanates is their toxicity as long as the curing process to form the Polyurethane is not fully completed. According to the rating of the European Chemical Agency (ECHA) two of the most relevant isocyanates such as 4,4′-Methylenediphenyl Diisocyanate and Toluene Diisocyanate are suspected to cause cancer. Additionally 4,4′-Methylenediphenyl Diisocyanate is rated as harmful if inhaled whereas Toluene Diisocyanate is rated as fatal if inhaled. Especially during processing, the workers are potentially exposed to the toxic Isocyanates which requires special training to the workers together with high standards with respect to the safety equipment.

With increasing awareness of chemicals which comes along with stricter regulations of the particular national chemical legislations, the demand for less dangerous—isocyanate-free—technologies is growing. However, in the field of plastics with food contact, there are further toxicological aspects to consider in addition to the risk during processing. For example, it must be ensured that ideally no component of the cured resin, which is toxic or adversely affect the taste respectively the quality of the food, migrate into the food during storage or usage. Depending on the exact hazardous profile of the dedicated migrating component, a very strict threshold for the specific component is defined for a specific application or application area.

Resin compositions based on multifunctional acetoacetate ester compounds and multifunctional acrylates are predominantly characterized by a good respectively user-friendly hazardous profile, so that the potential hazards for the worker respectively user are significantly reduced in comparison with the usage of common industrial isocyanate compounds as mentioned above. Furthermore the acetoacetate ester compounds are readily available by diketenization of the respective alcohols.

However, for certain applications in the food industry, it is a disadvantage of these systems that the production of multifunctional acetoacetate ester compounds by diketenization usually requires tertiary amines as catalysts, typical tertiary amine catalysts used in the diketenization are trimethylamine, DABCO or 4-dimethylaminopyridine. These tertiary amine catalysts remain in the acetoacetate ester compounds after their synthesis and thereby are also contained in any resin prepared with such acetoacetate ester compounds.

A removal of the catalyst at the end of the diketenization reaction is sometimes not possible or just to some extent. Furthermore, from an economic point of view, removal of the catalyst is always associated with higher costs which is not every time economically supported by the application. Thus, using common diketenezation catalyst there is always some risk that the catalyst remaining in the resin will migrate into the food or adversely affect the food in some way.

The instant invention concerns the synthesis of acetoacetate ester compound using an amino alcohol catalyst which can itself be covalently incorporated in the polymeric resin network when the acetoacetate ester compound is reacted with a polyamine or with a multifunctional acrylate in a polymerization reaction. The polymer can then be used for example in polymer applications where the migration of compounds into food or into pharmaceuticals or to the skin of a human or animal needs to be limited or even excluded.

In contrast to methods that employ common isocyanate based resin compositions such as resin compositions containing 4,4′-methylenediphenyl isocyanates (MDI) or toluene diisocyanates (TDI), the method of the invention utilizes a resin composition that does not comprise such harmful or toxic compounds or compounds which are suspected to cause cancer and is thus less toxic to the environment as well as to the workers.

Furthermore compared to using the commonly tertiary amines as catalyst in the diketenization for the production of the acetoacetate ester compound, the method of the invention uses amino alcohol catalysts which will itself be converted in an acetoacetate ester compound during the diketenezation process so that in the later polymerization reaction which provides the resin the diketenized amino alcohol catalyst will partake in polymerization reaction as the acetoacetate ester compound. In the polymerization of the acetoacetate ester compound with a multifunctional amine or multifunctional acrylate the deketenized amino alcohol catalysts will thereby be covalently bonded into the polymer backbone. This will lead to a significant reduction or even exclusion of any migration potential of the catalyst. Furthermore it was unexpected that the catalytic activity of amino alcohol catalyst in the diketenization is high even though the amino alcohol catalyst itself will be diketenized during the diketenization of the precursor of the acetoacetate ester compound.

### Abbreviations and Definitions

eq eq., equiv., equivalent; usually molar equivalent if not stated otherwise

### SUMMARY OF THE INVENTION

Method for the preparation of an acetoacetylated alcohol AA-ALC by a reaction REAC1 of
an alcohol ALC with diketene in the presence of an amino alcohol AMIALC as catalyst; wherein

the amino functionality of AMIALC is a tertiary, non-aromatic amino functional group; AMIALC is a mono- di or tri alcohol.

### DETAILED DESCRIPTION OF THE INVENTION

The invention provides a method for the preparation of an acetoacetylated alcohol AA-ALC by a reaction REAC1 of an alcohol ALC with diketene in the presence of an amino alcohol AMIALC as catalyst;

wherein

the amino functionality of AMIALC is a tertiary, non-aromatic amino functional group; AMIALC is a mono- di or tri alcohol.

The reaction product (i.e., acetoacetylated alcohol AA-ALC) is not separated from the catalyst, or from the reaction products of the catalyst under the reaction conditions of REAC1, such as AA-AMIALC. Thus, the reaction product obtained by REAC1 will be AA-ALC that contains AA-AMIALC as impurity. The reaction product AA-ALC containing said impurities can be used in the polymerization reactions further described herein without further purification.

AMIALC can be a compound of formula (AMIALC-I), (AMIALC-II), or (AMIALC-III);

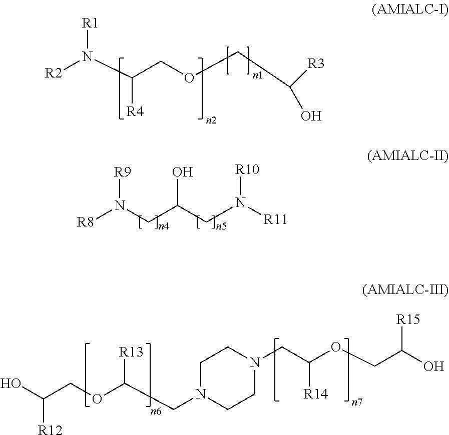

wherein

n1 is an integer from 1 to 18;

n2, n6 and n7 are identical or different and independently from each other an integer from 0 to 10;

n4 and n5 are identical or different and independently from each other an integer from 1 to 10;

R1, R2, R8, R9, R10 and R11 are identical or different and independently from each other C₁₋₁₀ alkyl, (CH₂CH₂O)ₙ₁₀H, or (CH(CH₃)CH₂)ₙ₁₁H;

R3, R12 and R15 are identical or different and independently from each other H, C₁₋₁₀ alkyl or CH₂—OH;

R4, R13 and R14 are identical or different and independently from each other H or CH₃;

n10 and n11 are identical or different and independently from each other an integer from 1 to 10.

Preferably,

n1 is an integer from 1 to 2;

n2, n6 and n7 are identical or different and independently from each other an integer from 0 to

5;

n4 and n5 are identical or different and independently from each other an integer from 1 to 5;

R1, R2, R8, R9, R10 and R11 are identical or different and independently from each other C₁₋₄ alkyl, (CH₂CH₂O)ₙ₁₀H, or (CH(CH₃)CH₂O)ₙ₁₁H;

R3, R12 and R15 are identical or different and independently from each other H, C₁₋₄ alkyl or CH₂—OH;

R4, R13 and R14 are identical or different and independently from each other H or CH₃;

n10 and n11 are identical or different and independently from each other an integer from 1 to 5;

more preferably,

n1 is an integer from 1 to 10;

n2, n6 and n7 are identical or different and independently from each other 0, 1 or 2;

n4 and n5 are identical or different and independently from each other 1 or 2;

R1, R2, R8, R9, R10 and R11 are identical or different and independently from each other C₁₋₄ alkyl, (CH₂CH₂O)ₙ₁₀H, or (CH(CH₃)CH₂O)ₙ₁₁H;

R3, R12 and R15 are identical or different and independently from each other H, C₁₋₂ alkyl or CH₂—OH;

R4, R13 and R14 are identical or different and independently from each other H or CH₃;

n10 and n11 are identical or different and independently from each other 1 or 2.

Embodiments of AMIALC can be selected from the group consisting of N,N-dibutyl ethanol amine, 1,3-bis(dimethylamino)-2-propanol, 1-(dimethylamino)-2-propanol,

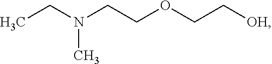

N,N′-piperazinediethanol, 2-dimethylaminoethanol, 3-(dimethylamino)-1,2-propanediol, N-methyldiethanolamine, triethanolamine, and mixtures thereof;

preferably, AMIALC is selected from the group consisting of N,N′-piperazinediethanol, 2-dimethylaminoethanol, 3-(dimethylamino)-1,2-propanediol, N-methyldiethanolamine, triethanolamine, and mixtures thereof;

more preferably, AMIALC is 2-dimethylaminoethanol, 3-(dimethylamino)-1,2-propanediol or N,N′-piperazinediethanol.

In a particularly preferred embodiment, AMIALC is 3-(dimethylamino)-1,2-propanediol or N,N′-piperazinediethanol.

The amount of AMIALC in REAC1 can be from 0.0001 to 0.1 eq, preferably from 0.0001 to 0.07 eq, more preferably from 0.0005 to 0.05 eq, even more preferably from 0.001 to 0.03 eq, the eq being based on the molar amount of ALC.

It has been surprisingly found that the catalytic activity of amino alcohol catalyst of the invention in the diketenization is high even though the amino alcohol catalyst itself will be diketenized during the diketenization of the precursor of the acetoacetate ester compound. Since the amino alcohol catalysts of the invention is converted into an acetoacetate ester compound AA-AMIALC during the diketenezation process, it will take part in the subsequent polymerization reaction of the AA-ALC with a multifunctional amine or multifunctional acrylate, and hence will be covalently bonded into the polymer backbone.

This is advantageous as it will lead to a significant reduction or even exclusion of any migration potential of the catalyst from the product. Therefore, the resulting plastics are particularly suited for use with, e.g., food products.

ALC can comprise 1, 2, 3, 4, 5 or 6 hydroxy functional groups.

Preferably, ALC comprises 1, 2, 3 or 4 hydroxy functional groups.

ALC can further comprise 1, 2, 3 or 4 tertiary, non-aromatic amino functional groups.

When ALC comprises one hydroxyl functional group, than ALC can be compound of formula (ALC-I) or compound of formula (ALC-II);

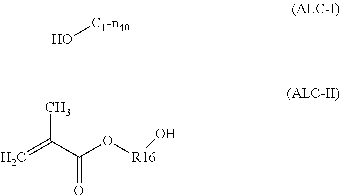

wherein

n40 is an integer from 2 to 30;

R16 is —(CH₂)ₙ₂₀—, —(CH₂CH₂O)ₙ₂₁CH₂CH₂—, or —(CH(CH₃)CH₂O)ₙ₂₂CH(CH₃)CH₂—;

n20 is an integer from 2 to 10;

n21 is an integer from 1 to 10;

n22 is an integer 0 to 10;

preferably,

n40 is an integer from 2 to 30;

R16 is —(CH₂)ₙ₂₀—, —(CH₂CH₂O)ₙ₂₁CH₂CH₂—, or —(CH(CH₃)CH₂O)ₙ₂₂CH(CH₃)CH₂—;

n20 is an integer from 2 to 6;

n21 is an integer from 1 to 5;

n22 is an integer 0 to 5;

more preferably,

n40 is an integer from 2 to 30;

R16 is —(CH₂)ₙ₂₀—, —(CH₂CH₂O)ₙ₂₁CH₂CH₂—, or —(CH(CH₃)CH₂O)ₙ₂₂CH(CH₃)CH₂—;

n20 is an integer from 2 to 4;

n21 is an integer from 1 to 3;

n22 is an integer 0 to 3;

or,

n40 is an integer from 3 to 30;

R16 is —(CH₂)ₙ₂₀—, —(CH₂CH₂O)ₙ₂₁CH₂CH₂—, or —(CH(CH₃)CH₂O)ₙ₂₂CH(CH₃)CH₂—;

n20 is an integer from 2 to 4;

n21 is an integer from 1 to 3;

n22 is an integer 0 to 3;

or,

n40 is an integer from 4 to 30;

R₁₆ is —(CH₂)ₙ₂₀—, —(CH₂CH₂O)ₙ₂₁CH₂CH₂—, or —(CH(CH₃)CH₂O)ₙ₂₂CH(CH₃)CH₂—;

n20 is an integer from 2 to 4;

n21 is an integer from 1 to 3;

n22 is an integer 0 to 3;

such as methanol, ethanol, 1-propanol, 2-propanol, 1-butanol, 2-butanol, 2-methylpropan-1-ol, 1,1-dimethyl ethanol, 3-methylbutan-1-ol, 2-methylbutan-1-ol, 2,2-dimethylpropan-1-ol, pentan-3-ol, pentan-2-ol, 3-methylbutan-2-ol, 2-methylbutan-2-ol, hexan-1-ol, hexan-2-ol, hexan-3-ol, 2-methylpentan-1-ol, 3-methylpentan-1-ol, 4-methylpentan-1-ol, 2-methylpentan-2-ol, 3-methylpentan-2-ol, 4-methylpentan-2-ol, 2-methylpentan-3-ol, 3-methylpentan-3-ol, 2,2-dimethylbutan-1-ol, 2,3-dimethylbutan-1-ol, 3,3-dimethylbutan-1-ol, 2,3-dimethylbutan-2-ol, 3,3-dimethylbutan-2-ol, 2-ethylbutan-1-ol, 1-heptanol, 2-heptanol, 3-heptanol, 4-heptanol, 1-octanol, 2-octanol, nonal-1-ol, decan-1-ol, undecan-1-ol, dodecan-1-ol, tridecan-1-ol, tetradecan-1-ol, pentadecan-1-ol, hexadecan-1-ol, octadecan-1-ol, hexacosan-1-ol, triacontan-1-ol, or (hydroxymethyl)acrylate

When ALC comprises 2 or more hydroxyl functional groups, then ALC is, for example, 1,4-butanediol, 1,6-hexanediol, neopentyl glycol, 4,8-bis(hydroxymethyl)tricyclo[5.2.1.02,6]decane, 2-methyl-1,3-propanediol, mono-, di-, tri- and tetraethylene glycol, polyethylene glycol, mono-, di-, tri- and tetrapropylene glycol, polypropylene glycol, cyclohexane dimethanol, trimethylolethane, trimethylolpropane, ethoxylated trimethylolethane, propoxylated trimethylolethane, ethoxylated trimethylolpropane, propoxylated trimethylolpropane, pentaerythritol, glycerol, and combinations thereof;

some further examples of suitable ALC include tetra-, penta-, and higher polyols (i.e., polyols with 4, 5, or more hydroxyl functional groups), including, for example, di(trimethylolpropane), dipentaerythritol, polyvinyl alcohols, polyester resins, decaglycerol deca-(Z)-oleate, and combinations thereof.

ALC can also be AMIALC.

When ALC is AMIALC, then the reaction product of REAC1 is an acetoacetylated AMIALC (AA-AMIALC). In this case AMIALC functions as its own catalyst in its own deketenization.

When ALC is AMIALC and AMIALC is 3-(dimethylamino)-1,2-propanediol, then AA-ALC is AA-AMIALC and AA-AMIALC is compound of formula (AA-DMAPD).

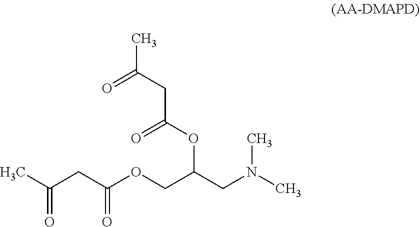 [SMILES: CC(=O)CC(=O)OCC(CN(C)C)OC(=O)CC(C)=O]

When ALC is AMIALC and AMIALC is N,N′-piperazinediethanol, then AA-ALC is AA-AMIALC and AA-AMIALC is compound of formula (AA-NNPDE).

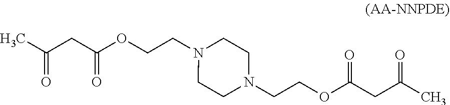 [SMILES: CC(=O)CC(=O)OCCN1CCN(CCOC(=O)CC(C)=O)CC1]

Embodiments of ALC are selected from the group consisting of neopentyl glycol, ethylene glycol, cyclohexanedimethanol, glycerol, trimethylolpropane, pentaerythritol, polyvinyl alcohols, polyester resins, AMIALC, and combinations thereof;

preferably, ALC is selected from the group consisting of trimethylolpropane, glycerol, ethylene glycol, AMIALC, and combinations thereof;

more preferably, ALC is trimethylolpropane, glycerol or AMIALC;

even more preferably, ALC is trimethylolpropane or AMIALC;

especially, ALC is glycerol or AMIALC;

with AMIALC as defined herein, also with all its embodiments.

AA-ALC according to the method of the invention comprises all possible acetoacetylated ALC which can be obtained by REAC1, AA-ALC comprises for example one, two, three, four, five or six acetoacetate ester functional groups. In another embodiment, AA-ALC comprises for example two, three, four, five or six acetoacetate ester functional groups. Preferably, AA-ALC comprises one, two, three or four acetoacetate ester functional groups. in another preferred embodiment, AA-ALC comprises two, three or four acetoacetate ester functional groups. The inventors have found that AA-ALC based on primary alcohols ALC generally exhibit faster curing and are thus advantageous in the method of the invention. AA-ALC is, for example,
acetoacetylated methanol, ethanol, 1-propanol, 2-propanol, 1-butanol, 2-butanol, 2-methylpropan-1-ol, 1,1-dimethylethanol, 3-methylbutan-1-ol, 2-methylbutan-1-ol, 2,2-dimethylpropan-1-ol, pentan-3-ol, pentan-2-ol, 3-methylbutan-2-ol, 2-methylbutan-2-ol, hexan-1-ol, hexan-2-ol, hexan-3-ol, 2-methylpentan-1-ol, 3-methylpentan-1-ol, 4-methylpentan-1-ol, 2-methylpentan-2-ol, 3-methylpentan-2-ol, 4-methylpentan-2-ol, 2-methylpentan-3-ol, 3-methylpentan-3-ol, 2,2-dimethylbutan-1-ol, 2,3-dimethylbutan-1-ol, 3,3-dimethylbutan-1-ol, 2,3-dimethylbutan-2-ol, 3,3-dimethylbutan-2-ol, 2-ethylbutan-1-ol, 1-heptanol, 2-heptanol, 3-heptanol, 4-heptanol, 1-octanol, 2-octanol, nonal-1-ol, decan-1-ol, undecan-1-ol, dodecan-1-ol, tridecan-1-ol, tetradecan-1-ol, pentadecan-1-ol, hexadecan-1-ol, octadecan-1-ol, hexacosan-1-ol, triacontan-1-ol, or (hydroxymethyl)acrylate;
1,4-butanediol diacetoacetate, 1,6-hexanediol diacetoacetate, neopentyl glycol diacetoacetate, the diacetoacetate of 4,8-bis(hydroxymethyl)tricyclo[5.2.1.0^{2,6}]decane, 2-methyl-1,3-propanediol diacetoacetate, mono-, di-, tri- and tetraethylene glycol diacetoacetate, polyethylene glycol diacetoacetate, mono-, di-, tri- and tetrapropylene glycol diacetoacetate, polypropylene glycol diacetoacetate, cyclohexane dimethanol diacetoacetate, trimethylolethane triacetoacetate, trimethylolpropane triacetoacetate, ethoxylated trimethylolethane triacetoactate, propoxylated trimethylolethane triacetoactate, ethoxylated trimethylolpropanetriacetoactate, propoxylated trimethylolpropanetriacetoactate, pentaerythritol triacetoacetate, glycerol triacetoacetate, and combinations thereof;
some further examples of suitable acetoacetate ester compounds include tetra-, penta-, and higher acetoacetates of polyols (i.e., polyols on which 4, 5, or more hydroxyl groups are linked to acetoacetate groups through ester linkages), including, for example, di(trimethylolpropane) tetraacetoacetate, pentaerythritol tetraacetoacetate, dipentaerythritol pentaacetoacetate, dipentaerythritol hexaacetoacetate, acetoacetylated polyvinyl alcohols, acetoacetylated polyester resins, decaglycerol deca-(Z)-oleate, and combinations thereof;
AA-AMIALC;

in a preferred embodiment, AA-ALC is selected from the group consisting of
acetoacetylated methanol, acetoacetylated ethanol, acetoacetylated 1-propanol, acetoacetylated 2-propanol, acetoacetylated 1-butanol, acetoacetylated 2-butanol, acetoacetylated 2-methylpropan-1-ol, acetoacetylated 1,1-dimethylethanol, acetoacetylated (hydroxymethyl)acrylate, neopentyl glycol diacetoacetate, ethylene glycol diacetoacetate, cyclohexanedimethanol diacetoacetate, glycerol triacetoacetate, trimethylolpropane triacetoacetate, pentaerythritol tetraacetoacetate, acetoacetylated polyvinyl alcohols, acetoacetylated polyester resins, AA-AMIALC, and combinations thereof;

in a more preferred embodiment, AA-ALC is selected from the group consisting of acetoacetylated methanol, acetoacetylated ethanol, acetoacetylated (hydroxymethyl)acrylate, trimethylolpropane triacetoacetate, glycerol triacetoacetate, ethylene glycol diacetoacetate, AA-AMIALC, and combinations thereof;

in an even more preferred embodiment, AA-ALC is acetoacetylated methanol, acetoacetylated ethanol, acetoacetylated (hydroxymethyl)acrylate, trimethylolpropane triacetoacetate, glycerol triacetoacetate, or AA-AMIALC;

in an especial embodiment, AA-ALC is acetoacetylated methanol, acetoacetylated ethanol, acetoacetylated (hydroxymethyl)acrylate, trimethylolpropane triacetoacetate or AA-AMIALC;

in another especial embodiment, AA-ALC is acetoacetylated methanol, acetoacetylated ethanol, acetoacetylated (hydroxymethyl)acrylate, glycerol triacetoacetate or AA-AMIALC;

in another especial embodiment, AA-ALC is acetoacetylated methanol, acetoacetylated (hydroxymethyl)acrylate, glycerol triacetoacetate or AA-AMIALC;

with AMIALC as defined herein, also with all its embodiments.

The molar amount of diketene in REAC1 can be from 0.9 to 1.5 fold, preferably from 0.94 to 1.5 fold, more preferably from 0.94 to 1.4 fold, even more preferably from 0.94 to 1.3 fold, especially from 0.94 to 1.1 fold, more especially from 0.94 to 1.05 fold, of the molar amount of ALC divided by the number of hydroxyl functional groups of ALC.

The reaction temperature TEMP1 of REAC1 can be from ambient temperature to 100° C., preferably from 30 to 80° C.

The reaction time TIME1 of REAC1 can be from 1 to 12 h, preferably from 2 to 10 h, more preferably from 4 to 9 h.

TIME1 can comprise the time of mixing diketene with ALC at TEMP1 and any time of e.g. stirring of the reaction mixture at TEMP1 after the mixing of diketene with ALC.

REAC1 can be done under ambient or elevated pressure.

REAC1 can be done under inert atmosphere.

AA-ALC can be isolated after REAC1 according to procedures known to the skilled person.

Further subject of the invention is a method for the preparation of a resin RESINCOMP by a polymerization reaction POLYMREAC of AA-ALC with an acrylate compound ACRYLCOMP, with an polyamine POLYAMI or with a mixture thereof;

wherein

AA-ALC has been prepared by REAC1;

with AA-ALC and REAC1 as defined herein, also with all its embodiments.

ACRYLCOMP to be used in the method of the invention comprises for example two, three, four, five or six acrylate functional groups. Preferably, ACRYLCOMP comprises two, three or four acrylate functional groups.

Suitable ACRYLCOMP is, for example, 1,4-butanediol diacrylate, 1,6-hexanediol diacrylate, neopentyl glycol diacrylate, the diacrylate of 4,8-bis(hydroxymethyl)tricyclo[5.2.1.02,6]decane, 2-methyl-1,3-propanediol diacrylate, mono-, di-, tri- and tetraethylene glycol diacrylate, polyethylene glycol diacrylate, mono-, di-, tri- and tetrapropylene glycol diacrylate, polypropylene glycol diacrylate, cyclohexane dimethanol diacrylate, alkoxylated hexanediol diacrylate, alkoxylated cyclohexane dimethanol diacrylate, propoxylated neopentyl glycol diacrylate, glycerol triacrylate, trimethylolethane triacrylate, trimethylolpropane triacrylate, ethoxylated trimethylolethane triacrylate, propoxylated trimethylolethane triacrylate, ethoxylated trimethylolpropanetriacrylate, propoxylated trimethylolpropanetriacrylate, pentaerythritol triacrylate, pentaerythritol tetraacrylate, dipentaerythritol pentaacrylate, di(trimethylolpropane) tetraacrylate, acrylated polyester oligomer, bisphenol A diacrylate, ethoxylated bisphenol A diacrylate, tris(2-hydroxyethyl) isocyanurate triacrylate, acrylated aliphatic urethane oligomer, acrylated aromatic urethane oligomer, or the like, or combinations thereof.

In a preferred embodiment, ACRYLCOMP is selected from the group consisting of 1,6-hexanediol diacrylate, tetraethylene glycol diacrylate, tripropylene glycol diacrylate, trimethylolpropane triacrylate, pentaerythritol triacrylate, pentaerythritol tetraacrylate, di(trimethylolpropane) tetraacrylate, and combinations thereof.

In a more preferred embodiment, ACRYLCOMP is selected from the group consisting of tripropylene glycol diacrylate, trimethylolpropane triacrylate, di(trimethylolpropane) tetraacrylate, and combinations thereof.

In an even more preferred embodiment, ACRYLCOMP is trimethylolpropane triacrylate.

Preferably, the term “ethoxylated” means a total content of 1 to 10 ethylenoxide units per molecule.

Preferably the term “propoxylated” means a total content of 1 to 10 propylenoxide units per molecule.

POLYAMI to be used in the method of the invention comprises for example two, three, four, five or six primary amino functional groups. Preferably, POLYAMI comprises two, three or four primary amino functional groups.

POLYAMI can be a compound selected from the group consisting of compound of formula (POLYAMI-I), a compound of formula (POLYAMI-II), a compound of formula (POLYAMI-III), compound of formula (POLYAMI-IV), or a compound of formula (POLYAMI-V);

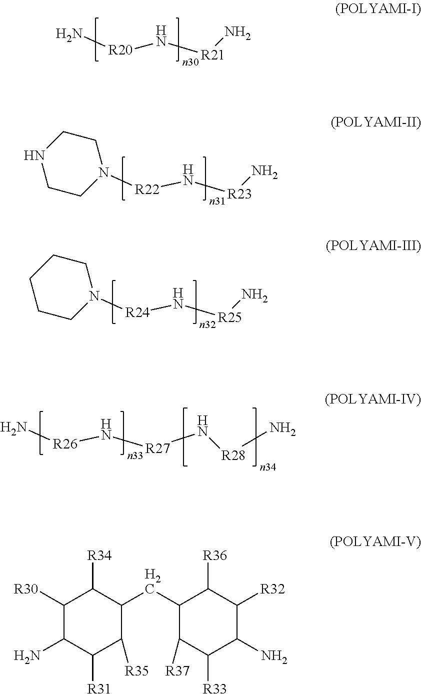

wherein

R20, R21, R22, R23, R24 and R25 are identical or different and independently from each other selected from the group consisting of —CH₂CH₂— and —CH(CH₃)CH₂—;

R26, R27 and R28 are identical or different and independently from each other —CH₂CH₂—, —CH₂CH₂CH₂— or —CH(CH₃)CH₂—;

n30, n31, n32, n33 and n34 are identical or different and independently from each other an integer from 0 to 10;

R30, R31, R32, R33, R34, R35, R36 and R37 are identical or different and independently from each other selected from the group consisting of H, methyl, ethyl, iso-propyl and chloro;

with the proviso, that

at least one of R30, R31, R34 and R35 is H; and

with the proviso, that

at least one of R32, R33, R36 and R37 is H;

preferably,

R20, R21, R22, R23, R24 and R25 are identical or different and independently from each other selected from the group consisting of —CH₂CH₂— and —CH(CH₃)CH₂—;

R26, R27 and R28 are identical or different and independently from each other —CH₂CH₂— or —CH₂CH₂CH₂—;

n30, n31, n32, n33 and n34 are identical or different and independently from each other an integer from 0 to 5;

more preferably,

R20, R21, R22, R23, R24 and R25 are —CH₂CH₂—;

R26, R27 and R28 are identical or different and independently from each other —CH₂CH₂— or —CH(CH₃)CH₂—;

n30, n31, n32, n33 and n34 are identical or different and independently from each other an integer from 0 to 5.

Specific embodiments of POLYAMI are 1,2-bis(3-aminopropylamino)ethane and 4,4′-methylenebis(2-methylcyclohexylamine).

The amount of AA-ALC in RESINCOMP may vary.

The amount of AA-ALC in POLYMREAC with ACRYLCOMP may range from 20 to 70 wt %, preferably from 20 to 60 wt %, more preferably from 20 to 55 wt %, the wt % being based in the total weight of RESINCOMP.

The amount of ACRYLCOMP in RESINCOMP may vary. The amount of ACRYLCOMP may range from 30 to 80 wt %, preferably from 40 to 80 wt %, more preferably from 45 to 80 wt %, the wt % being based in the total weight of RESINCOMP.

The amount of AA-ALC in POLYMREAC with POLYAMI may range from 30 to 80 wt %, preferably from 35 to 80 wt %, the wt % being based in the total weight of RESINCOMP.

The amount of POLYAMI in RESINCOMP may vary. The amount of POLYAMI may range from 20 to 70 wt %, preferably from 25 to 65 wt %, the wt % being based in the total weight of RESINCOMP.

When POLYMREAC is a polymerization reaction of AA-ALC with ACRYLCOMP, then a tertiary amine curing catalyst AMINCAT may be present during POLYMREAC.

POLYMREAC begins with the mixing of AA-ALC and ACRYLCOMP and any AMINCAT or with the mixing of AA-ALC with POLYAMI.

POLYMREAC is also called a curing of RESINCOMP or can comprise a curing of RESINCOMP. POLYMREAC or said curing of RESINCOMP provides RESINCOMP in form of a resin, preferably in form of a cured or partially cured resin.

The pKa of the protonated amine species of AMINCAT may vary over a broad range. Generally, stronger bases, i.e., AMINCAT with greater pKa values provide a higher catalytic activity during the curing reaction. The pKa of AMINCAT is at least 8, or at least 10, or at least 11, or at least 12. Preferably, the pKa of the tertiary amine curing catalyst is from 10 to 15, more preferably from 11 to 14, even more preferably from 12 to 14. As used herein, pKa refers to the pKa of the corresponding conjugate base in water.

AMINCAT may be selected from the group consisting of 1,1,3,3-tetramethylguanidine (TMG), 1,4-diazabicyclo[2.2.2]octane (DABCO), triethylamine (TEA), N,N-dimethylisopropylamine, 1,8-Diazabicyclo(5.4.0)undec-7-ene (DBU), 1,5-Diazabicyclo(4.3.0)non-5-ene (DBN), and mixtures thereof.

Preferably, AMINCAT is selected from the group consisting of TMG, DABCO, DBU, DBN, and mixtures thereof.

More preferably, AMINCAT is selected from the group consisting of TMG, DABCO, and mixtures thereof.

Even more preferably, AMINCAT is TMG.

in another even more preferable embodiment, AMINCAT is DABCO.

The amount of AMINCAT in RESINCOMP may vary. Generally, the curing time of a fiber reinforced part produced according to the invention depends on the amount of AMINCAT that is used. Therefore, the amount of AMINCAT can be varied to adapt the method of the invention to different applications and needs. In the method of the invention, the amount of AMINCAT may range from 0.01 to 10 wt %, from 0.05 to 7.5 wt %, from 0.05 to 5 wt %, from 0.05 to 2.5 wt %, from 0.05 to 1 wt %, from 0.05 to 0.75 wt %, from 0.075 to 0.5 wt %, or from 0.1 to 0.3 wt %, the wt % being based in the total weight of RESINCOMP.

In an especial embodiment, AA-ALC is selected from the group consisting of neopentyl glycol diacetoacetate, ethylene glycol diacetoacetate, cyclohexanedimethanol diacetoacetate, glycerol triacetoacetate, trimethylolpropane triacetoacetate, pentaerythritol tetraacetoacetate, acetoacetylated polyvinyl alcohols, acetoacetylated polyester resins, and combinations thereof;

ACRYLCOMP is selected from the group consisting of 1,6-hexanediol diacrylate, tetraethylene glycol diacrylate, tripropylene glycol diacrylate, trimethylolpropane triacrylate, pentaerythritol triacrylate, pentaerythritol tetraacrylate, di(trimethylolpropane) tetraacrylate, and combinations thereof and

AMINCAT comprises TMG, DABCO or a mixture thereof, preferably, AMINCAT comprises TMG.

In a more especial embodiment,

AA-ALC is selected from the group consisting of trimethylolpropane triacetoacetate, glycerol triacetoacetate, ethylene glycol diacetoacetate, and combinations thereof;

ACRYLCOMP is selected from the group consisting of tripropylene glycol diacrylate, trimethylolpropane triacrylate, di(trimethylolpropane) tetraacrylate, and combinations thereof; and

AMINCAT comprises TMG, DABCO or a mixture thereof, preferably, AMINCAT comprises TMG.

In an even more especial embodiment,

AA-ALC comprises trimethylolpropane triacetoacetate or glycerol triacetoacetate;

ACRYLCOMP comprises trimethylolpropane triacrylate; and

AMINCAT comprises TMG, DABCO or a mixture thereof, preferably, AMINCAT comprises TMG.

In a particular embodiment,

AA-ALC comprises trimethylolpropane triacetoacetate;

ACRYLCOMP comprises trimethylolpropane triacrylate; and

AMINCAT comprises TMG, DABCO or a mixture thereof, preferably, AMINCAT comprises TMG.

In another particular embodiment,

AA-ALC comprises glycerol triacetoacetate;

ACRYLCOMP comprises trimethylolpropane triacrylate; and

AMINCAT comprises TMG, DABCO or a mixture thereof, preferably, AMINCAT comprises TMG.

The functional group ratio FGR AA/ACR of the functional groups AA of AA-ALC and ACR of ACRYLCOMP, as well as the functional group ratio FGR AA/AM of the functional groups AA of AA-ALC and AM of POLYAMI may vary, depending on the molar ratio of AA-ALC and of ACRYLCOMP or POLYAMI respectively as well as on the number of functional groups in each compound comprised in RESINCOMP.

FGR of RESINCOMP may be calculated using the following formula in case of POLYMREAC between AA-ALC and ACRYLCOMP:

FGR=[(n[mol]AA-ALC)×(number N of functional groups AA per AA-ALC)]/[(n[mol]ACRYLCOMP)×(number N of functional groups ACR per ACRYLCOMP)]

FGR of RESINCOMP may be calculated using the following formula in case of POLYMREAC between AA-ALC and POLYAMI:

FGR=[(n[mol]AA-ALC)×(number N of functional groups AA per AA-ALC)]/[(n[mol]POLYAMI)×(number N of functional groups AM per POLYAMI)]

The calculation of FGRs for specific AA-ALC and ACRYLCOMP ratios is further exemplified in the Table A.

TABLE A
FGRs of exemplary resin formulations for the case of
POLYMREAC between AA-ALC and ACRYLCOMP.

| AA-ALC | | ACRYLCOMP | | | Functional |
|---|---|---|---|---|---|
| N | | N | | | Group |
| Functional | n | Functional | n | | Ratio |
| Groups | [mol] | Groups | [mol] | Molar Ratio | AA/ACR |
| 2 | 1 | 2 | 1 | 1:1 | 1 |
| 2 | 1 | 2 | 2 | 1:2 | 0.5 |
| 3 | 1 | 3 | 1 | 1:1 | 1 |
| 3 | 1 | 3 | 2 | 1:2 | 0.5 |
| 3 | 1 | 2 | 1 | 1:1 | 1.5 |
| 3 | 1 | 2 | 3 | 1:3 | 0.5 |
| 3 | 2 | 4 | 3 | 2:3 | 0.5 |

FGRs of exemplary resin formulations for the case of POLYMREAC between AA-ALC and POLYAMI can be calculated analogously.

In a preferred embodiment of the invention, the FGR AA/ACR is from 0.15 to 1.50, preferably from 0.2 to 1.25, more preferably from 0.25 to 0.95, even more preferably from 0.25 to 0.85, especially from 0.3 to 0.85, more especially from 0.3 to 0.75, even more especially from 0.3 to 0.6.

In a preferred embodiment of the invention, the FGR AA/AM is from 0.5 to 1.50, preferably from 0.6 to 1.4, more preferably from 0.7 to 1.3, even more preferably from 0.8 to 1.2, especially from 0.9 to 1.1, more especially from 0.95 to 1.05.

In an especial embodiment, AA-ALC is selected from the group consisting of neopentyl glycol diacetoacetate, ethylene glycol diacetoacetate, cyclohexanedimethanol diacetoacetate, glycerol triacetoacetate, trimethylolpropane triacetoacetate, pentaerythritol tetraacetoacetate, acetoacetylated polyvinyl alcohols, acetoacetylated polyester resins, and combinations thereof;

ACRYLCOMP is selected from the group consisting of 1,6-hexanediol diacrylate, tetraethylene glycol diacrylate, tripropylene glycol diacrylate, trimethylolpropane triacrylate, pentaerythritol triacrylate, pentaerythritol tetraacrylate, di(trimethylolpropane) tetraacrylate, and combinations thereof;

AMINCAT comprises TMG, DABCO or a mixture thereof, preferably, AMINCAT comprises TMG; and

FGR is from 0.3 to 0.75.

In a more especial embodiment,

AA-ALC is selected from the group consisting of trimethylolpropane triacetoacetate, glycerol triacetoacetate, ethylene glycol diacetoacetate, and combinations thereof;

ACRYLCOMP is selected from the group consisting of tripropylene glycol diacrylate, trimethylolpropane triacrylate, di(trimethylolpropane) tetraacrylate, and combinations thereof;

AMINCAT comprises TMG, DABCO or a mixture thereof, preferably, AMINCAT comprises TMG; and

FGR is from 0.3 to 0.75.

In an even more especial embodiment,

AA-ALC comprises trimethylolpropane triacetoacetate or glycerol triacetoacetate;

ACRYLCOMP comprises trimethylolpropane triacrylate;

AMINCAT comprises TMG, DABCO or a mixture thereof, preferably, AMINCAT comprises TMG;

FGR is from 0.3 to 0.75.

In a particular embodiment,

AA-ALC comprises trimethylolpropane triacetoacetate;

ACRYLCOMP comprises trimethylolpropane triacrylate;

AMINCAT comprises TMG, DABCO or a mixture thereof, preferably, AMINCAT comprises TMG;

FGR is from 0.3 to 0.75.

In another particular embodiment,

AA-ALC comprises glycerol triacetoacetate;

ACRYLCOMP comprises trimethylolpropane triacrylate;

AMINCAT comprises TMG, DABCO or a mixture thereof, preferably, AMINCAT comprises TMG;

FGR is from 0.3 to 0.75.

Preferably, when POLYMREAC is a polymerization reaction of AA-ALC with an acrylate compound ACRYLCOMP, then AA-ALC, ACRYLCOMP and AMINCAT are in a first step mixed to provide RESINCOMP. The mixing can be done in any order:

in one embodiment EMBA of POLYMREAC at first ACRYLCOMP is premixed with AMINCAT to provide a premix PREMIXA, then PREMIXA is mixed with AA-ALC to provide RESINCOMP;

in another embodiment EMBB of POLYMREAC at first AA-ALC is premixed with AMINCAT to provide a premix PREMIXB, then PREMIXB is mixed with ACRYLCOMP to provide RESINCOMP;

in another embodiment EMBC of POLYMREAC at first AA-ALC is premixed with ACRYLCOMP to provide a premix PREMIXC, then PREMIXC is mixed with AMINCAT to provide RESINCOMP.

Preferred order of mixing are EMBA and EMBB, more preferably EMBA.

AA-ALC and/or ACRYLCOMP and/or POLYAMI and/or AMINCAT may be dissolved in a suitable non-aqueous solvent prior to mixing with each other. Suitable non-aqueous solvents are known to the skilled person and include, for example, acetone, ethyl acetate and alcohols, such as methanol, ethanol, butanol, isopropanol, and the like. If solvents are used in the method of the invention, the solvents may be evaporated from the RESINCOMP prior to curing.

Preferably, AA-ALC and ACRYLCOMP and POLYAMI are liquid at room temperature.

Preferably, AMINCAT is liquid at room temperature.

Preferably, RESINCOMP is liquid at room temperature right after mixing of the components.

Preferably, RESINCOMP is essentially free of solvent.

Preferably, no solvent is used for the preparation of RESINCOMP.

In particular, AA-ALC and ACRYLCOMP and POLYAMI, preferably also AMINCAT, are liquid at room temperature, RESINCOMP are essentially free of solvent, and no solvent is used for the preparation of RESINCOMP.

Alternatively, the viscosity of constituents of RESINCOMP itself may be decreased by using thinners that are consumed during the curing reaction and therefore only have a minor, or essentially no impact on the hardness or strength of the resulting fiber reinforced part. Suitable thinners include mono-functional acetoacetate ester compounds with low viscosity, such as C₁₋₄ alkyl acetoacetate e.g. methyl acetoacetate, ethyl acetoacetate, isopropyl acetoacetate, tert-butyl acetoacetate; preferably methyl acetoacetate. Preferably, a thinners is used in an amount of from 0.1 to 20 wt %, more preferably of from 0.1 to 10 wt %, even more preferably of from 0.2 to 5 wt %, the wt % based on the weight of RESINCOMP. Thus, in one embodiment, RESINCOMP further comprises a thinner, preferably methyl acetoacetate.

The mixing of the components of RESINCOMP to provide RESINCOMP can be done at various temperatures, such as from 0 to 50° C., preferably from 0 to 40° C.; more preferably from 0 to 30° C., even more preferably from 5 to 30° C., especially from 5° C. to room temperature.

In one embodiment of the invention, RESINCOMP is degassed before it is cured. Degassing is preferably done by applying vacuum to RESINCOMP. The vacuum that is applied to RESINCOMP is preferably below 50 mbar, more preferably below 30 mbar, even more preferably below 10 mbar.

POLYMREAC and any curing of RESINCOMP may be done in a wide range of temperatures, such as a temperature of from 0 to 200° C., preferably from 0 to 160° C., more preferably from 0 to 140° C., even more preferably of from 5 to 140° C., especially of from 5 to 130° C., more especially of from 5 to 120° C., even more especially of from 5 to 110° C.

The time of POLYMREAC and the time of any curing of RESINCOMP depends on various parameters, such as the temperature which is chosen for the reaction and for any curing, the amount and the type of RESINCOMP which needs to be cured, the amount and the type of AMINCAT, therefore the time for curing can vary in a wide range, such as from 5 sec to 48 h, or 5 sec to 24 h, or from 5 sec to 16 h.

A further subject of the invention is a kit for forming RESINCOMP to be used in the method for preparation of the resin RESINCOMP by a polymerization reaction POLYMREAC, with RESINCOMP and POLYMREAC as defined herein, also with all its embodiments, the kit comprises
AA-ALC, ACRYLCOMP, and AMINCAT; or
AA-ALC and POLYAMI; or
PREMIXA and AA-ALC; or
PREMIXB and ACRYLCOMP; or
PREMIXC and AMINCAT;

with PREMIXA, PREMIXB, PREMIXC, AA-ALC, ACRYLCOMP, POLYAMI and AMINCAT as defined herein, also with all their embodiments.

A further subject of the invention is RESINCOMP, with RESINCOMP as defined herein, also with all its embodiments.

A further subject of the invention is RESINCOMP obtainable by the method for preparation RESINCOMP as described herein, also with all its embodiments.

Further subject of the invention is the use of RESINCOMP for the manufacture of parts, coatings, films, foams, adhesives; and the use of RESINCOMP as a part, a coating, a film, a foam, or an adhesives; with RESINCOMP as defined herein, also with all its embodiments.

Further subject of the invention is compound of formula (AA-DMAPD) and compound of formula (AA-NNPDE).

### EXAMPLES

### Abbreviations Used in this Specification and Materials

¹H(q)-NMR ¹H Quantitative Nuclear Magnetic Resonance Spectroscopy
AA acetoacetyl, acetoacetate, acetoacetyl functional group, acetoacetylated
AA-groups acetoacetyl functional groups
AA-DMAE acetoacetylated DMAE
AA-DMAPD bis acetoacetylated 3-(dimethylamino)-1,2-propanediol, bis acetoacetylated DMAPD, compound of formula (AA-DMAPD)
AA-NNPDE bis acetoacetylated N,N′-piperazinediethanol, bis acetoacetylated NNPDE, compound of formula (AA-NNPDE)
AA-TMP acetoacetoxylated TMP
AA-TMP (×1) mono-acetoacetoxylated TMP
AA-TMP (×2) bis-acetoacetoxylated TMP
AA-TMP (×3) tris-acetoacetoxylated TMP

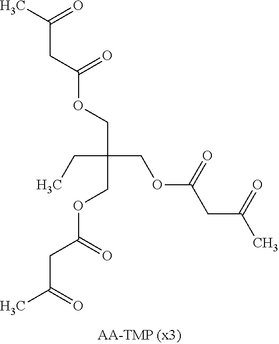 [SMILES: CCC(COC(=O)CC(C)=O)(COC(=O)CC(C)=O)COC(=O)CC(C)=O]

ACR acrylate, acrylate functional group
ACR-groups acrylate functional group
AM primary amino group, primary amino functional group
AM-groups primary amino functional group
BAPAE 1,2-bis(3-aminopropylamino)ethane, technical grade 94%, CAS 10563-26-5, Sigma Aldrich, Switzerland

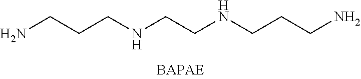 [SMILES: NCCCNCCNCCCN]

Bisphenol A diglycidyl ether CAS 1675-54-3, D3415 of Sigma Aldrich, Switzerland

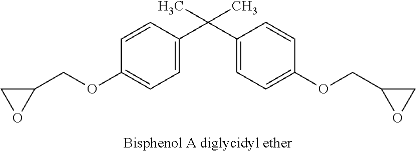 [SMILES: CC(C)(c1ccc(OCC2CO2)cc1)c1ccc(OCC2CO2)cc1]

Diketene diketene, CAS 674-82-8, Lonza Ltd, 3930 Visp, Switzerland
DMAE 2-dimethylaminoethanol, CAS 108-01-0, Sigma Aldrich, Switzerland

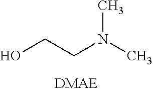

DMAPD 3-(dimethylamino)-1,2-propanediol, CAS 623-57-4, Sigma Aldrich, Switzerland

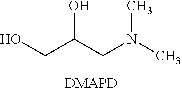

M-MCHA 4,4′-methylenebis(2-methylcyclohexylamine), CAS 6864-37-5, Sigma Aldrich, Switzerland

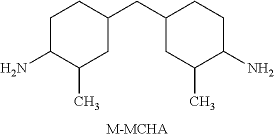 [SMILES: CC1CC(CC2CCC(N)C(C)C2)CCC1N]

NMDEA N-methyldiethanolamine, CAS 105-59-9, Sigma Aldrich, Switzerland

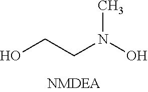

NNPDE N,N′-piperazinediethanol, CAS 122-96-3, Fluorochem, UK

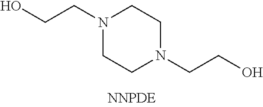 [SMILES: OCCN1CCN(CCO)CC1]

TEtA triethanolamine, CAS 102-71-6

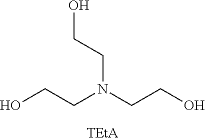

TMG 1,1,3,3-tetramethylguanidine; CAS 80-70-6, Lonza Ltd, 3930 Visp, Switzerland
TMP 1,1,1-tris(hydroxymethyl)propane, CAS 77-99-6, Sigma Aldrich, Switzerland

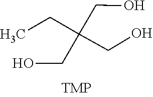

TMPTA trimethylolpropane triacrylate, CAS 15625-89-5, Laromer® TMPTA, BASF, 67056 Ludwigshafen, Germany
wt % weight percent, percent by weight

Methods

Glass Transition Temperature Tg

The glass transition temperature T_{g} was measured by Thermal Mechanical Analysis (TMA). The machine used was a Mettler Toledo instrument TMA SDTA840. The sample dimensions were 6 times 6 mm² (length times width) and 4 to 4.5 mm thickness. The test method applied two heating ramps (first ramp: 25 to 120° C. at 10 K/min and a second ramp 25 to 150° C. at 10 K/min). The T_{g} was evaluated on the second ramp.

Shore a Hardness

Shore A hardness values were measured with a digital CV Shore Durometer (www.bowersgroup.co.uk).

Needle used: D 0.79 mm with 35°

Loading forces: 1.812 lb (822 g)

Maximum penetration is 0.097 to 0.1 inch (2.5 to 2.54 mm)

The durometer was standing on a flat solid surface. The measurement was done at ambient temperature.

1. The sample was placed on the base of the durometer

2. The position of the sample was changed so that the needle of the gauge was at least 8 to 10 mm from the edge of the sample

3. The sample was made sure not to wiggle and not to stand skewed.

4. With the lever on the right side of the gauge needle, the needle was pressed against the surface of the sample. The lever was pushed the whole way down to the stopper, in order to ensure the complete weight has been applied onto the gauge needle.

5. The result has been checked on the gauge display. The result has been recorded after the number of the gauge had settled to a stable value.

6. The measurement has been repeated 3 times at different spots which have been at least 6 mm away from each other.

7. The average Shore A value has been calculated from these 3 measured values and reported.

### Example 1: Acetoacetoxylation of TMP Using NNPDE

TMP (100.0 g, 745 mmol, 1 equiv.) and NNPDE (0.390 g, 2.24 mmol, 0.003 equiv.) were charged into a reactor, the mixture was then heated to 65° C. until all solid had been molten. Then diketene (186.7 g, 2220 mmol, 2.98 equiv.) was added in 5 h while maintaining the reaction temperature at 70 to 75° C. Then the mixture was stirred for 2 h at 70 to 75° C. when no further heat release was observed. Then the reactor content was uncharged to afford 282.0 g of an orange viscous product mixture.

¹H-NMR confirmed the formation of AA-TMP (×3) and AA-TMP (×2).

Based on ¹H(q)-NMR, 82% yield for AA-TMP (×3) and 14% for AA-TMP (×2) was determined.

The content of acetoacetoxy functional groups was 62%, which corresponds to 94% diketene conversion.

### Example 2: Acetoacetoxylation of TMP Using DMAE

TMP (150.0 g, 1118 mmol, 1 equiv.) and DMAE (0.300 g, 3.354 mmol, 0.003 equiv.) were charged into a reactor, the mixture was then heated to 65° C. until all solid had been molten. Then diketene (282.2 g, 3354 mmol, 3.00 equiv.) was added in 5 h while maintaining the reaction temperature at 70° C. Then the mixture was stirred for 2 h at 70° C. when no further heat release was observed. Then the reactor content was uncharged to afford 423.0 g of an orange viscous product mixture.

¹H-NMR confirmed the formation of AA-TMP (×3) and AA-TMP (×2).

Based on ¹H(q)-NMR, 85% yield for AA-TMP (×3) and 10% for AA-TMP (×2) was determined.

The content of acetoacetoxy functional groups was 63%, which corresponds to 95% diketene conversion.

### Example 3: Acetoacetoxylation of TMP Using DMAPD

TMP (150.0 g, 1118 mmol, 1 equiv.) and DMAPD (0.400 g, 3.354 mmol, 0.003 equiv.) were charged into a reactor, the mixture was then heated to 65° C. until all solid had been molten. Then diketene (282.2 g, 3354 mmol, 3.00 equiv.) was added in 5 h while maintaining the reaction temperature at 70° C. Then the mixture was stirred for 3 h at 70° C. when no further heat release was observed. Then the reactor content was uncharged to afford 421.0 g of an orange viscous product mixture.

¹H-NMR confirmed the formation of AA-TMP (×3) and AA-TMP (×2).

Based on ¹H(q)-NMR, 67% yield for AA-TMP (×3) and 24% for AA-TMP (×2) was determined.

The content of acetoacetoxy functional groups was 58%, which corresponds to 87% diketene conversion.

### Example 4: Acetoacetoxylation of TMP Using NMDEA

TMP (137.0 g, 1000 mmol, 1 equiv.) and NMDEA (1.20 g, 1.00 mmol, 0.01 equiv.) were charged into a reactor, the mixture was then heated to 65° C. until all solid had been molten. Then diketene (252.0 g, 3000 mmol, 3.00 equiv.) was added in 2.1 h while maintaining the reaction temperature at 70 to 75° C. Then the mixture was stirred for 3.5 h at 70° C. when no further heat release was observed. Then the reactor content was uncharged to afford 383.9 g of an orange viscous product mixture.

1H-NMR confirmed the formation of AA-(×3)-TMP and AA-(×2)-TMP.

Based on 1H(q)-NMR, 45% yield for AA-(×3)-TMP and 50% for AA-(×2)-TMP was determined.

The content of acetoacetoxy functional groups was 58%, which corresponds to 88% diketene conversion.

### Example 5: Acetoxyacetylation of NNPDE

A reactor was charged with NNPDE (104.5 g, 600 mmol, 1 equiv.) and acetone (418.2 g), a suspension formed which was then heated to 40° C. Then diketene (104.5 g, 1243 mmol, 2.07 equiv.) was added to the stirred suspension within 3 h while maintaining the reaction temperature at 40 to 45° C. Then the mixture was stirred for 3 h at 55° C. when no further heat release was observed. The mixture was concentrated under vacuum at 50° C. by removal of acetone to afford 198.0 g of a yellow viscous liquid, which solidified upon standing at ambient temperature.

¹H-NMR confirmed the formation of bis acetoacetoxylated NNPDE, that is compound of formula (AA-NNPDE), while no unreacted starting material was detected.

Based on ¹H(q)-NMR, 94% yield for bis acetoacetoxylated NNPDE was determined.

### Example 6: Acetoxyacetylation of DMAE

A reactor was charged with DMAE (207.4 g, 2327 mmol, 1 equiv.) and was heated to 70° C. Then diketene (195.6 g, 2327 mmol, 1 equiv.) was added within 4 h while maintaining the reaction temperature at 70 to 71° C. Then the mixture was stirred for 1 hour at 70° C. when no further heat release was observed. The resultant mixture was uncharged, yielding 393 g of a brownish and clear liquid.

¹H-NMR confirmed the formation of 2-(dimethylamino)ethyl 3-oxobutanoate, while no unreacted starting material was detected.

Based on ¹H(q)-NMR, 99% yield for 2-(dimethylamino)ethyl 3-oxobutanoate (AA-DMAE) was determined.

### Example 7: Curing of AA-DMAE with TMPTA and TMG

AA-DMAE (5.53 g, 30.3 mmol AA-groups), prepared according to example 6, was added to a mixture of TMPTA (6.47 g, 58.1 mmol ACR-groups) and TMG (1 wt % based on the weight of the mixture of AA-DMAE, TMPTA and TMG) at ambient temperature. After mixing the formulation was transferred to a round aluminum dish (diameter d=6.5 cm). After 3 h, a yellow disc was obtained.

Tg onset (TMA): 42° C.

Shore A: 80

### Example 8: Curing of AA-NNPDE with M-MCHA

AA-NNPDE (5.14 g, 29.0 mmol AA-groups), prepared according to example 5, was melted at 40° C., the melt was then add to a mixture of M-MCHA (6.86 g, 57.4 mmol NH₂ groups) and TMG (1 wt % based on the weight of the mixture of AA-NNPDE, M-MCHA and TMG).

After mixing the formulation was transferred to a round aluminum dish (diameter d=6.5 cm).

After 10 min, a yellow disc was obtained.

Tg onset (TMA): not determinable due to melting

Shore A: 94

### Example 9: Curing of AA-NNPDE with TMPTA and TMG

AA-NNPDE (4.24 g, 23.9 mmol AA-groups), prepared according to example 5, was melted at 40° C., the melt was then add to a mixture of TMPTA (7.76 g, 69.8 mmol ACR-groups) and TMG (1 wt % based on the weight of the mixture of AA-NNPDE, TMPTA and TMG). After mixing the formulation was transferred to a round aluminum dish (diameter d=6.5 cm). After 1 h, a yellow disc was obtained.

Tg onset (TMA): 44° C.

Shore A: 97

### Example 10: Reaction of AA-DMAE with BAPAE and Subsequent Curing with Epoxid Resin

AA-DMAE (4.98 g, 27.3 mmol AA groups), prepared according to example 6 was added to BAPAE (2.38 g, 27.3 mmol NH₂ groups), the mixture was shaken, whereby an instant heat-release was observed. After 60 min at ambient temperature a viscous clear orange mixture was obtained.

¹H-NMR analysis confirmed the quantitative enamine formation.

Then Bisphenol A diglycidyl ether (4.64 g, 27.3 mmol) was added to the viscous clear orange mixture, with shaking a homogenous mixture was obtained which was transferred to a round aluminum dish (diameter d=6.5 cm). After 24 h, an orange disc was obtained.

### Example 11: Curing in the Presence of TMG of TMPTA with AA-TMP Produced with DMAE

AA-TMP (4.68 g, 34.5 mmol AA-groups), prepared according to example 2 was added to a mixture of TMPTA (7.32 g, 65.7 mmol ACR-groups) and TMG (1 wt % based on the weight of the mixture of AA-TPM, TMPTA and TMG) at ambient temperature. After mixing the formulation was transferred to a round aluminum dish (diameter d=6.5 cm). After 10 min, a yellow disc was obtained.

Tg onset (TMA): 65° C.

Shore A: 96

### Example 12: Curing with M-MCHA of AA-TMP Produced with DMAE

AA-TMP, prepared according to example 2, was added to M-MCHA at room temperature. After mixing the formulation was transferred to a round aluminum dish (diameter d=6.5 cm). After 10 min, a yellow disc was obtained.

1. A method for the preparation of an acetoacetylated alcohol AA-ALC by a reaction REAC1 of an alcohol ALC with diketene in the presence of an amino alcohol AMIALC as catalyst;
wherein
the amino functionality of AMIALC is a tertiary, non-aromatic amino functional group;
AMIALC is a mono- di or tri alcohol; and
ALC is selected from the group consisting of neopentyl glycol, ethylene glycol, cyclohexanedimethanol, glycerol, trimethylolpropane, pentaerythritol, polyvinyl alcohols, polyester resins, AMIALC, and combinations thereof.

2. The method according to claim 1, wherein
AMIALC is a compound of formula (AMIALC-I), (AMIALC-II), or (AMIALC-III); 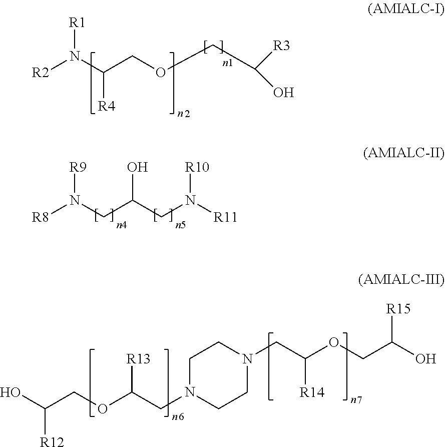
wherein
n1 is an integer from 1 to 18;
n2, n6 and n7 are identical or different and independently from each other an integer from 0 to 10;
n4 and n5 are identical or different and independently from each other an integer from 1 to 10;
R1, R2, R8, R9, R10 and R11 are identical or different and independently from each other C₁₋₁₀ alkyl, (CH₂CH₂O)ₙ₁₀H, or (CH(CH₃)CH₂O)ₙ₁₁H;
R3, R12 and R15 are identical or different and independently from each other H, C₁₋₁₀ alkyl or CH₂—OH;
R4, R13 and R14 are identical or different and independently from each other H or CH₃;
n10 and n11 are identical or different and independently from each other an integer from 1 to 10.

3. The method according to claim 1, wherein
n1 is an integer from 1 to 2;
n2, n6 and n7 are identical or different and independently from each other an integer from 0 to 5;
n4 and n5 are identical or different and independently from each other an integer from 1 to 5;
R1, R2, R8, R9, R10 and R11 are identical or different and independently from each other C₁₋₄ alkyl, (CH₂CH₂O)ₙ₁₀H, or (CH(CH₃)CH₂O)ₙ₁₁H;
R3, R12 and R15 are identical or different and independently from each other H, C₁₋₄ alkyl or CH₂—OH;
R4, R13 and R14 are identical or different and independently from each other H or CH₃;
n10 and n11 are identical or different and independently from each other an integer from 1 to 5.

4. The method according to claim 1, wherein
AMIALC is selected from the group consisting of N,N-dibutyl ethanol amine, 1,3-bis(dimethylamino)-2-propanol, 1-(dimethylamino)-2-propanol, 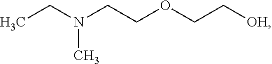
N,N′-piperazinediethanol, 2-dimethylaminoethanol, 3-(dimethylamino)-1,2-propanediol, N-methyldiethanolamine, triethanolamine, and mixtures thereof.

5-18. (canceled)

19. A compound of formula (AA-DMAPD).

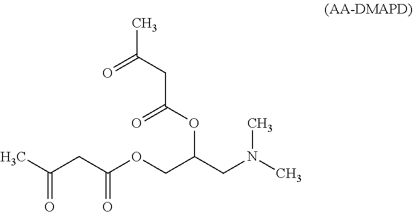 [SMILES: CC(=O)CC(=O)OCC(CN(C)C)OC(=O)CC(C)=O]

20. A compound of formula (AA-NNPDE).

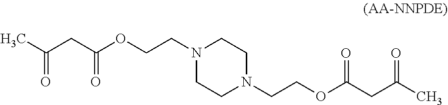 [SMILES: CC(=O)CC(=O)OCCN1CCN(CCOC(=O)CC(C)=O)CC1]

21. A method for the preparation of a resin RESINCOMP, the method comprising the steps of:
a) the preparation of an acetoacetylated alcohol AA-ALC by a reaction REAC1 of an alcohol ALC with diketene in the presence of an amino alcohol AMIALC as catalyst; wherein
the amino functionality of AMIALC is a tertiary, non-aromatic amino functional group;
AMIALC is a mono- di or tri alcohol;
ALC is selected from the group consisting of neopentyl glycol, ethylene glycol, cyclohexanedimethanol, glycerol, trimethylolpropane, pentaerythritol, polyvinyl alcohols, polyester resins, AMIALC, and combinations thereof; and
b) a polymerization reaction POLYMREAC of AA-ALC obtained in step a) with an acrylate compound ACRYLCOMP, with a polyamine POLYAMI or with a mixture thereof.

22. The method according to claim 21, wherein
AMIALC is a compound of formula (AMIALC-I), (AMIALC-II), or (AMIALC-III); 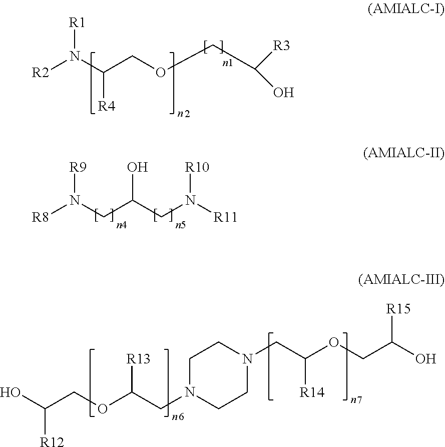
wherein
n1 is an integer from 1 to 18;
n2, n6 and n7 are identical or different and independently from each other an integer from 0 to 10;
n4 and n5 are identical or different and independently from each other an integer from 1 to 10;
R1, R2, R8, R9, R10 and R11 are identical or different and independently from each other C₁₋₁₀ alkyl, (CH₂CH₂O)ₙ₁₀H, or (CH(CH₃)CH₂O)ₙ₁₁H;
R3, R12 and R15 are identical or different and independently from each other H, C₁₋₁₀ alkyl or CH₂—OH;
R4, R13 and R14 are identical or different and independently from each other H or CH₃;
n10 and n11 are identical or different and independently from each other an integer from 1 to 10.

23. The method according to claim 21, wherein n1 is an integer from 1 to 2;
n2, n6 and n7 are identical or different and independently from each other an integer from 0 to 5;
n4 and n5 are identical or different and independently from each other an integer from 1 to 5;
R1, R2, R8, R9, R10 and R11 are identical or different and independently from each other C₁₋₄ alkyl, (CH₂CH₂O)ₙ₁₀H, or (CH(CH₃)CH₂O)ₙ₁₁H;
R3, R12 and R15 are identical or different and independently from each other H, C₁₋₄ alkyl or CH₂—OH;
R4, R13 and R14 are identical or different and independently from each other H or CH₃;
n10 and n11 are identical or different and independently from each other an integer from 1 to 5.

24. The method according to claim 21, wherein
AMIALC is selected from the group consisting of N,N-dibutyl ethanol amine, 1,3-bis(dimethylamino)-2-propanol, 1-(dimethylamino)-2-propanol, 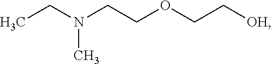
N,N′-piperazinediethanol, 2-dimethylaminoethanol, 3-(dimethylamino)-1,2-propanediol, N-methyldiethanolamine, triethanolamine, and mixtures thereof.

25-27. (canceled)

28. The method according to claim 21, wherein
ACRYLCOMP is 1,4-butanediol diacrylate, 1,6-hexanediol diacrylate, neopentyl glycol diacrylate, the diacrylate of 4,8-bis(hydroxymethyl)tricyclo[5.2.1.02,6]decane, 2-methyl-1,3-propanediol diacrylate, mono-, di-, tri- and tetraethylene glycol diacrylate, polyethylene glycol diacrylate, mono-, di-, tri- and tetrapropylene glycol diacrylate, polypropylene glycol diacrylate, cyclohexane dimethanol diacrylate, alkoxylated hexanediol diacrylate, alkoxylated cyclohexane dimethanol diacrylate, propoxylated neopentyl glycol diacrylate, glycerol triacrylate, trimethylolethane triacrylate, trimethylolpropane triacrylate, ethoxylated trimethylolethane triacrylate, propoxylated trimethylolethane triacrylate, ethoxylated trimethylolpropanetriacrylate, propoxylated trimethylolpropanetriacrylate, pentaerythritol triacrylate, pentaerythritol tetraacrylate, dipentaerythritol pentaacrylate, di(trimethylolpropane) tetraacrylate, acrylated polyester oligomer, bisphenol A diacrylate, ethoxylated bisphenol A diacrylate, tris(2-hydroxyethyl) isocyanurate triacrylate, acrylated aliphatic urethane oligomer, acrylated aromatic urethane oligomer, or combinations thereof.

29. The method according to claim 21, wherein
POLYAMI is a compound selected from the group consisting of compound of formula (POLYAMI-I), a compound of formula (POLYAMI-II), a compound of formula (POLYAMI-III), compound of formula (POLYAMI-IV), or a compound of formula (POLYAMI-V); 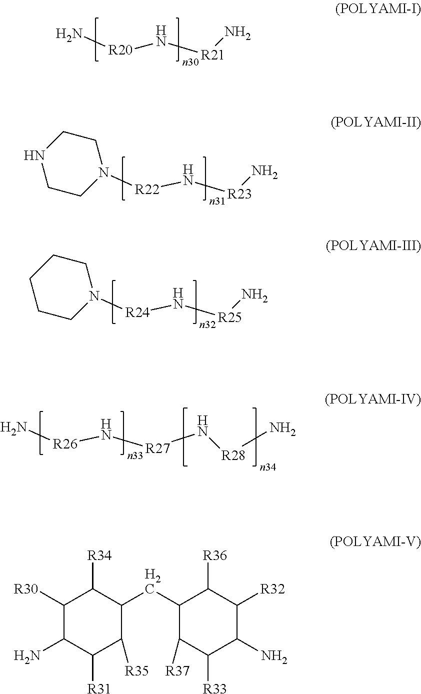
wherein
R20, R21, R22, R23, R24 and R25 are identical or different and independently from each other selected from the group consisting of —CH₂CH₂— and —CH(CH₃)CH₂—;
R26, R27 and R28 are identical or different and independently from each other —CH₂CH₂—, —CH₂CH₂CH₂— or —CH(CH₃)CH₂—;
n30, n31, n32, n33 and n34 are identical or different and independently from each other an integer from 0 to 10;
R30, R31, R32, R33, R34, R35, R36 and R37 are identical or different and independently from each other selected from the group consisting of H, methyl, ethyl, iso-propyl and chloro;
with the proviso, that
at least one of R30, R31, R34 and R35 is H; and
with the proviso, that
at least one of R32, R33, R36 and R37 is H.

30. The method according to claim 29, wherein
R20, R21, R22, R23, R24 and R25 are identical or different and independently from each other selected from the group consisting of —CH₂CH₂— and —CH(CH₃)CH₂—;
R26, R27 and R28 are identical or different and independently from each other —CH₂CH₂— or —CH₂CH₂CH₂—;
n30, n31, n32, n33 and n34 are identical or different and independently from each other an integer from 0 to 5.

31. The method according to claim 21, wherein
POLYMREAC is a polymerization reaction of AA-ALC with ACRYLCOMP and a tertiary amine curing catalyst AMINCAT is present during POLYMREAC.

32. The method according to claim 31, wherein
the pKa of the protonated amine species of AMINCAT is at least 8.

33. The method according to claim 31, wherein
AMINCAT is selected from the group consisting of 1,1,3,3-tetramethylguanidine (TMG), 1,4-diazabicyclo[2.2.2]octane (DABCO), triethylamine (TEA), N,N-dimethylisopropylamine, 1,8-Diazabicyclo(5.4.0)undec-7-ene (DBU), 1,5-Diazabicyclo(4.3.0)non-5-ene (DBN), and mixtures thereof.

34. The method according to claim 31, wherein
a) EMBA of POLYMREAC at first ACRYLCOMP is premixed with AMINCAT to provide a premix PREMIXA, then PREMIXA is mixed with AA-ALC to provide RESINCOMP;
b) EMBB of POLYMREAC at first AA-ALC is premixed with AMINCAT to provide a premix PREMIXB, then PREMIXB is mixed with ACRYLCOMP to provide RESINCOMP; or
c) EMBC of POLYMREAC at first AA-ALC is premixed with ACRYLCOMP to provide a premix PREMIXC, then PREMIXC is mixed with AMINCAT to provide RESINCOMP.

35. A kit for forming RESINCOMP to be used in the method for the preparation of a resin RESINCOMP according to claim 2,
the kit comprising
AA-ALC, an acrylate compound ACRYLCOMP, and a tertiary amine curing catalyst AMINCAT; or
AA-ALC and a polyamine POLYAMI; or
PREMIXA and AA-ALC; or
PREMIXB and an acrylate compound ACRYLCOMP; or
PREMIXC and a tertiary amine curing catalyst AMINCAT;
wherein
EMBA of POLYMREAC at first ACRYLCOMP is premixed with AMINCAT to provide premix PREMIXA,
EMBB of POLYMREAC at first AA-ALC is premixed with AMINCAT to provide premix PREMIXB and
EMBC of POLYMREAC at first AA-ALC is premixed with ACRYLCOMP to provide a premix PREMIXC; and
AA-ALC is prepared by a reaction REAC1 of an alcohol ALC with diketene in the presence of an amino alcohol AMIALC as a catalyst, wherein
AMIALC is a mono- di or tri alcohol; and
ALC is selected from the group consisting of neopentyl glycol, ethylene glycol, cyclohexanedimethanol, glycerol, trimethylolpropane, pentaerythritol, polyvinyl alcohols, polyester resins, AMIALC, and combinations thereof.

36. A RESINCOMP prepared according to the method of claim 21.

37. The use of RESINCOMP according to claim 36 for the manufacture of parts, coatings, films, foams, adhesives.

38. The use of RESINCOMP according to claim 36 as a part, a coating, a film, a foam, or an adhesives.
